(19) United States
(12) Patent Application Publication
(10) Pub. No.: US 2024/0316014 A1
(43) Pub. Date: Sep. 26, 2024

(54) METHOD FOR TREATING DEMYELINATING CONDITIONS

(71) Applicants: ACADEMIA SINICA, Taipei City (TW); GWO XI STEM CELL APPLIED TECHNOLOGY CO., LTD., Zhubei City (TW)

(72) Inventors: Joyce Jean LU, Taipei City (TW); Hsiao-Chun Huang, Taipei City (TW); Pei-Lun La, New Taipei City (TW); Chi-Hou Ng, Taipei City (TW); Ming-Hsi Chuang, Hsinchu County (TW)

(73) Assignees: ACADEMIA SINICA, Taipei City (TW); GWO XI STEM CELL APPLIED TECHNOLOGY CO., LTD., Zhubei City (TW)

(21) Appl. No.: 18/126,273

(22) Filed: Mar. 24, 2023

(51) Int. Cl. A61K 31/44; A61K 9/00; A61K 31/352; A61K 31/404; A61P 25/00

(52) U.S. Cl. CPC A61K 31/44; A61K 9/0019; A61K 9/0085; A61K 31/352; A61K 31/404; A61P 25/00

(57) ABSTRACT

The present invention generally relates to a method for treating a demyelinating condition, in particular, by administering a combination comprising therapeutically effective amounts of a Rho-associated protein kinase (ROCK) inhibitor, a cyclin-dependent kinase (CDK) inhibitor, and a cyclic adenosine monophosphate (cAMP) activator to a subject in need thereof.

### TECHNOLOGY FIELD

The present invention generally relates to a method for treating a demyelinating condition, in particular, by administering a combination comprising therapeutically effective amounts of a Rho-associated protein kinase (ROCK) inhibitor, a cyclin-dependent kinase (CDK) inhibitor, and a cyclic adenosine monophosphate (cAMP) activator to a subject in need thereof.

### BACKGROUND OF THE INVENTION

Oligodendrocytes, also named as oligodendroglia, are a type of neuroglia that generates myelin sheaths that wrap around axons in the CNS to create an electrical insulation that increases the rate of electrical conduction [1,2]. Generally, one oligodendrocyte can wrap about 50 axons and increases the travel rate of nerve impulses to about 100 times faster in myelinated axons as compared to non-myelinated neurons [3]. In addition, oligodendrocytes help nerve regeneration by providing nutrients and an appropriate environment [4-7]. During development, oligodendrocytes are derived from oligodendrocyte progenitor cells (OPCs), which are distributed in the central nervous system and can repair demyelinated axons to form new myelin sheaths [8,9]. Therefore, promoting the recruitment or differentiation of OPCs is considered to be the main strategy for enhancing myelination or in the pursuit of developing novel treatments against demyelinating diseases [10].

Myelin loss or dysfunction affects about 2-2.5 million people worldwide [11]. Multiple sclerosis (MS) is one of the most common chronic demyelinating diseases caused by the loss of oligodendrocytes when the immune system attacks the myelin sheath [12-14]. Damaged demyelinated areas in the CNS contribute to the development of MS symptoms including difficulties in, or damage to vision, bladder, memory/thinking, pain, cramps, speech problems, and swallowing. Besides, MS can also cause stroke, muscle weakness, or death [15]. Several studies injected OPCs into the damaged areas to ensheath the demyelinated region which have yielded promising results [16-18]. However, it is always hard to obtain sufficient human primary OPCs for cell therapy [16-18]. Therefore, producing enough OPCs or promoting remyelination by glial cells is a key issue of concern. Currently, human embryonic stem cells (hESCs) and induced pluripotent stem cells (iPSCs) provide a source material for the production of OPCs through the ectopic expression of transcription factors accompanied by soluble factors to promote differentiation [19-21]. Studies have shown that neural stem cells (NSCs) derived from hESCs or OPCs derived from mesenchymal stem cells (MSCs) can improve the symptoms of myelin damage in animal experiments [22-24]. However, OPCs produced by viral transduction have a higher risk of insertion mutations, and the differentiation process of ESCs and iPSCs is laborious and takes about 60-100 days; the process of differentiation is also costly and has the risk of teratoma formation [25-29].

Liu et al. used a chemical approach to convert mouse fibroblasts into induced OPCs for cell therapy. [30]. Tsui et al. induced OPCs from rat MSCs as cell material to treat demyelination [24]. However, cell therapy may still suffer from issues of immune rejection and the low rates of survival of transplanted cells in vivo. Therefore, there is a need to provide a cell-free therapy for demyelination

### SUMMARY OF THE INVENTION

In this invention, it is surprisingly found that a combination comprising a Rho-associated protein kinase (ROCK) inhibitor, a cyclin-dependent kinase (CDK) inhibitor, and a cyclic adenosine monophosphate (cAMP) activator can effectively rescue demyelination in vivo by administration to a demyelinating lesion of a subject in need thereof.

Therefore, the present invention provides a method for treating a demyelinating condition in a subject in need thereof, comprising administering to the subject therapeutically effective amounts of a ROCK inhibitor, a CDK inhibitor, and a cAMP activator.

In some embodiments, the amounts of the ROCK inhibitor, the CDK inhibitor and the cAMP inhibitor are effective in increasing a level of remyelination and/or decreasing a level of demyelination in the subject.

In some embodiments, the ROCK inhibitor, the CDK inhibitor and the cAMP inhibitor are administrated into a demyelinating lesion in the subject where myelin is lost or damaged.

In some embodiments, the ROCK inhibitor, the CDK inhibitor and the cAMP inhibitor are injected to the brain of the subject.

In some embodiments, the ROCK inhibitor, the CDK inhibitor and the cAMP inhibitor are small molecules.

In some embodiments, the ROCK inhibitor is Y27632 or a pharmaceutically acceptable salt, the CDK inhibitor is SU9516 or a pharmaceutically acceptable salt, and the cAMP inhibitor is forskolin or a pharmaceutically acceptable salt.

In some embodiments, the ROCK inhibitor, the CDK inhibitor and the cAMP inhibitor are administered simultaneously or sequentially.

In some embodiments, the demyelinating condition is an inflammatory demyelinating condition.

In some embodiments, the demyelinating condition occurs in a disease selected from the group consisting of multiple sclerosis (MS), acute hemorrhagic inflammatory disease (AHL), cerebral palsy, acute-disseminated encephalomyelitis (ADEM), central pontine myelinolysis, progressive multifocal leukoencephalopathy, congenital leukodystrophies, Parkinson's disease, Huntington's disease, schizophrenia.

Also provided is a pharmaceutical composition comprising therapeutically effective amounts of a ROCK inhibitor, a CDK inhibitor, and a cAMP activator and a pharmaceutically acceptable carrier. A pharmaceutical composition as described herein is useful for treating a demyelinating condition in a subject in need thereof.

Further provided is use of a combination of a ROCK inhibitor, a CDK inhibitor, and a cAMP activator for manufacturing a medicament for treating a demyelinating condition in a subject in need thereof.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A-1D show that high-throughput screening of small molecules for conversion of fibroblasts into oligodendrocyte-like cells. (FIG. 1A) Fibroblasts change cell morphology after treatment with different chemicals. Phase-contrast images represent solvent control (fibroblasts) (top left), Y27632-treated fibroblasts (top right), SU9516-treated fibroblasts (bottom left), and fibroblasts treated with a combination of Y27632 and SU9516 (bottom right). (FIG. 1B) Immunofluorescence staining shows the oligodendrocyte-specific marker 04 on induced cells (red). DAPI (blue) was used to stain nuclei. (FIG. 1C) 1,540 chemicals were tested, and the candidate molecules that promoted reprogramming were screened based on Y27632 and SU9516 after valproic acid (VPA) treatment. These images show different chemical combination treatments of fibroblasts. Forskolin (FSK) treated cells maintain dome-shaped cell morphology and dendritic structures. The ingredients of ‘VYSF’ includes valproic acid, Y27632, SU9516, and forskolin. (FIG. 1D) Testing of VPA. Pre-treated and co-treated VPA with ‘YSF’ cocktails. Fibroblasts were treated with VPA for 2 days then treated with the YSF mixture for 1 day or fibroblasts were co-treated with VPA and YSF mixture for 1 day. Phase-contrast images representative of solvent control (fibroblasts) (left), VPA pre-treatment (middle), and VPA combination treatment (right) of cells are shown. VYSF refers to valproic acid, Y27632, SU9516, and forskolin, respectively.

FIG. 2 shows the sketch of iOLCs generation process. The flow chart shows Y27632, SU9516, and FSK treatment with induction medium one day after VPA pre-treatment and then two days of treatment that converts fibroblasts into iOLCs.

FIG. 3 shows expression of oligodendrocyte markers observed by immunofluorescence on iOLCs induced with four chemicals. iOLCs induced by the VYSF cocktail are compared to the solvent control (fibroblasts) group. 1×10⁴ fibroblasts were seeded in each well of 24 well-plates, after two days of expansion, fibroblasts were treated with the VYSF cocktail. Data showing protein expression, including oligodendrocyte-specific transcription factor Olig2 (upper left, green); oligodendrocyte-specific surface antigens 04 (upper-middle, red) and O1 (upper right, red); oligodendrocyte-specific markers MBP (bottom left, red), GPR17 (bottom middle, red) and galactosylceramidase (bottom right, red). DAPI (blue) was used to stain nuclei. VYSF refers to valproic acid, Y27632, SU9516, and forskolin, respectively.

FIGS. 4A-4C show marker expression in chemically induced oligodendrocyte-like cells. iOLCs are the cells treated with valproic acid, Y27632, SU9516 and forskolin (VYSF). (FIG. 4A) The mRNA expression of myelin-forming proteins MBP and PLP1 between the VYSF-treated group (iOLCs) and the solvent control group were confirmed by quantitative real-time polymerase chain reaction (qRT-PCR). Data are presented as mean #standard deviation. (n=3). GAPDH was used as reference gene. Significant differences were determined using Student's t-test, *p<0.05, ** p<0.005, *** p<0.001. (FIG. 4B) Western blot analysis showing transcription factor Nkx2.2 protein up-regulation in the VYSF-treated group (iOLCs). Quantification of Western blot data is shown at the bottom. Data are presented as mean±standard deviation. (n=5). β-actin was used as an internal control. Significant differences were determined using Student's t-test, *p<0.05. (FIG. 4C) Flow cytometry was performed to quantify A2B5+ cells of chemical cocktail VYSF-treated fibroblasts (iOLCs). In the chemically treated group, A2B5+ was detectable in an average of 50% of the cells. Data are presented as mean±standard deviation. (n=3). Significant differences were determined using Student's t-test, *p<0.05.

FIG. 5 shows the sketch of remyelination improvement with injected 3 chemical (3C) cocktail into a cuprizone-induced demyelination mouse model. The flow chart shows that the 3C cocktail (Y27632, SU9516 and FSK) and PBS were injected into mice in the corpus callosum after 12 weeks of cuprizone-induced demyelination. After 2 weeks of recovery, 3C cocktail (Y27632, SU9516 and FSK) injection groups, PBS injection groups and positive control groups (normal diet) are sacrificed for the follow-up analysis.

FIGS. 6A-6B show that chemical cocktail injection rescues cuprizone-induced demyelination in mice. Induction of demyelination in B6 mice by cuprizone. The YSF-cocktail and PBS were injected directly into the corpus callosum of mice, then the mice were allowed to recover for two weeks before dissection. YSF refers to Y27632, SU9516 and forskolin. (FIG. 6A) Luxol fast blue staining was performed to detect the remyelination process in the corpus callosum. The quantification of Luxol fast blue was compared between the YSF-cocktail and PBS injected groups after 2 weeks of recovery before dissection. Data are presented as mean±standard deviation. (n=6). Significant differences were determined by ANOVA followed by Sidak's multiple comparison test, *p<0.05, ** p<0.005, *** p<0.001, **** p<0.0001. (FIG. 6B) After 2 weeks of recovery, immunofluorescence staining was performed to observe the protein expression of the YSF-cocktail and PBS-injected groups. Comparison of myelination densities for MBP (top) and PLP (bottom) protein expression in the corpus callosum of the YSF-cocktail and PBS-injected groups. DAPI (blue) was used to stain nuclei.

FIGS. 7A-7B shows decrease of g-ratio after chemical cocktail injection into the corpus callosum. B6 mice suffered induced demyelination by consuming a cuprizone diet. The YSF-cocktail and PBS were injected directly into the corpus callosum of mice and then the mice were allowed to recover for two weeks before dissection. Chemical treatment refers to Y27632, SU9516, and forskolin (YSF). (FIG. 7A) Electron microscope images of YSF-cocktail and PBS injected groups. (FIG. 7B) G-ratios are calculated from YSF-cocktail and PBS injected groups of mice brain slices. Data are presented as mean±standard deviation. Significant differences were determined using Student's t-test, *p<0.05, ** p<0.005, *** p<0.001, **** p<0.0001.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

The term “about” as used herein means plus or minus 10% of the numerical value of the number with which it is being used. Therefore, about 1% means in the range of 0.9% to 1.1%.

As used herein, the term “demyelinating condition” refers to a condition characterized by loss or damage of myelin sheath. Myelin is a lipid-rich material that surrounds nerve cell axons to form myelin sheath which protects nerve cells, prevent ion leakage and keep them maintain their function. A demyelinating condition may have a demyelinating lesion in the myelin sheet that surrounds nerve fibers in the brain, optic nerves, and spinal cord. The destruction of the protective myelin sheet may be caused by viral, autoimmune or genetic diseases and can lead to symptoms such as difficulty with cognitive and motor activities. In particular, a demyelinating condition is an inflammatory demyelinating condition where the immune system plays a role in destroying myelin. Specifically, a demyelinating condition may occur in, for example, multiple sclerosis (MS), acute hemorrhagic inflammatory disease (AHL), cerebral palsy, acute-disseminated encephalomyelitis (ADEM), central pontine myelinolysis, progressive multifocal leukoencephalopathy, congenital leukodystrophies, Parkinson's disease, Huntington's disease, schizophrenia. A level of demyelination can be expressed by myelin g-ratio which is a relative measurement of myelin thickness, defined as the ratio of the inner to outer radius of the myelin sheath. Specifically, a g-ratio of 1 means a completely naked axon without myelin while a g-ratio less than 1 means increased myelin sheath thickness. Namely, a lower g-ratio indicates that the axons are covered by thicker myelin sheaths (a decreased level of demyelinating) while a higher g-ratio indicates that the axons are covered by thinner myelin sheaths (an increased level of demyelinating).

Remyelination is a natural repair process to restore myelin sheaths upon damage. Remyelination involves activation, proliferation and migration of oligodendrocyte progenitor cells (OPCs). A level of remyelination can be measured in a pathological section by pale myelin staining, such as Luxol fast blue staining. Enhancement of remyelination is beneficial to restore a demyelinating condition.

The term “small molecule” as used herein refers to organic or inorganic molecules either synthesized or found in nature, generally having a molecular weight less than 10,000 grams per mole, particularly less than 5,000 grams per mole, particularly less than 2,000 grams per mole, particularly less than 1,000 grams per mole, for example, about 500 grams per mole, about 400 grams per mole and 300 grams per mole. In some embodiments, a small molecule refers to a non-polymeric, e.g. non-protein or nucleic acid based, chemical molecule.

As used herein, the term “pharmaceutically acceptable salt” includes acid addition salts. “Pharmaceutically acceptable acid addition salts” refer to those salts which retain the biological effectiveness and properties of the free bases, which are formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid and the like. The term “pharmaceutically acceptable salt” also includes base addition salts such as alkali metal salts e.g. lithium (Li), sodium (Na), and potassium (K) salts, alkaline earth metal salts e.g. calcium (Ca) and magnesium (Mg) salts, or salts of organic bases e.g. lysine, arginine, guanidine, diethanolamine, and choline, ammonium or substituted ammonium salts.

As used herein, a Rho-associated protein kinase (ROCK) inhibitor can refer to an agent that downregulates, decreases or suppresses the amount and/or activity of Rho-associated protein kinase. Examples of ROCK inhibitors as described herein include, but are not limited to, Y-27632, AS 1892802, GSK 269962, GSK 429286, H 1152 dihydrochloride, HA 1100 hydrochloride, OXA 06 dihydrochloride, RKI 1447 dihydrochloride, SB 772077B dihydrochloride, etc.

As used herein, a cyclin-dependent kinase (CDK) inhibitor can refer to an agent that downregulates, decreases or suppresses the amount and/or activity of cyclin-dependent kinase. Examples of CDK inhibitors as described herein include, but are not limited to, SU9516, PD-0332991, Roscovitine, SNS-032, Dinaciclib, Flavopiridol, AT7519, Flavopiridol, JNJ-7706621, AZD5438, MK-8776, PHA-793887, BS-181, Palbociclib (PD0332991) Isethionate, A-674563, abemaciclib, BMS-265246, PHA-767491, Milciclib, R547, NU6027, P276-00, MSC2530818, Senexin A, LY2857785, LDC4297, ON123300, Kenpaullone, K03861, THZ1 2HCl, AT7519 HCl, Purvalanol A, Ro-3306, XL413, LDC000067, ML167, TG003, Ribociclib, Wogonin, BIO, AZD1080, 1-Azakenpullone, and others, and a pharmaceutically acceptable salt thereof.

As used herein, a cyclic adenosine monophosphate (cAMP) activator can refer to an agent that increases intracellular levels of cAMP as compared to the background physiological intracellular level when the agent is absent. Examples of cAMP activators include, but are not limited to, forskolin, rolipram, NKH477, PACAP1-27, PACAP1-38 and others, and a pharmaceutically acceptable salt thereof.

As used here, the term “subject” as used herein includes human and non-human animals such as companion animals (such as dogs, cats and the like), farm animals (such as cows, sheep, pigs, horses and the like), or laboratory animals (such as rats, mice, guinea pigs and the like).

As used herein, the term “treating” when relating to therapeutically treating refers to the application or administration of a composition including one or more active agents to a subject afflicted with a disorder, a symptom or conditions of the disorder, or a progression of the disorder, with the purpose to cure, heal, alleviate, relieve, alter, remedy, ameliorate, improve, or affect the disorder, the symptoms or conditions of the disorder, the disabilities induced by the disorder, or the progression of the disorder.

As used herein, the term “therapeutically effective amount” used herein refers to the amount of an active ingredient to confer a therapeutic effect in a treated subject. For example, an effective amount for treating a demyelinating condition can be an amount that can decrease a level of demyelination and/or increase a level of remyelination in a subject. A level of demyelination and/or a level of remyelination can be determined and evaluated using methods known in the art, for example, by measuring g-ratio for a level of demyelination and an area by Luxol fast blue staining for a level of remyelination. The therapeutically effective amount may change depending on various reasons, such as administration route and frequency, body weight and species of the individual receiving said pharmaceutical, and purpose of administration.

According to the present invention, a combination of a ROCK inhibitor, a CDK inhibitor, and a cAMP activator is effective in rescuing demyelination in vivo.

Therefore, the present invention provides a method for treating a demyelination condition in a subject in need thereof, comprising administering to the subject therapeutically effective amounts of a combination comprising a ROCK inhibitor, a CDK inhibitor, and a cAMP activator. In particular, the present invention provides a cell-free therapy by directly administering a combination of a ROCK inhibitor, a CDK inhibitor and a cAMP inhibitor to a subject in need thereof. In the present invention, there is no need to obtain oligodendrocyte progenitor cells (OPCs) or prepare induced OPCs in vitro and then transfer the cells to the subject in need thereof for cell therapy.

Specifically, the ROCK inhibitor, the CDK inhibitor and the cAMP inhibitor are small molecules. Examples of a ROCK inhibitor, a CDK inhibitor, and a cAMP activator are described as above. Some particular examples are provided as follows.

TABLE A

| Name | Activity | Structure |
|---|---|---|
| Y-27632 | ROCK | Trans-4-[(1R)-1-Aminoethyl]-N-(4- |
| | inhibitor | pyridinyl)cyclohexanecarboxamide |
| | | 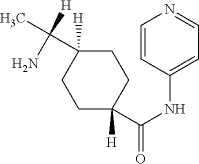 [SMILES: C[C@@H](N)[C@H]1CC[C@H](C(=O)Nc2ccncc2)CC1] |
| SU9516 | CDK | (Z)-3-((1H-imidazol-5-yl)methylene)-5- |
| | inhibitor | methoxyindolin-2-one |
| | | 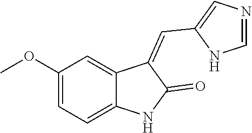 [SMILES: COc1ccc2c(c1)/C(=C/c1cnc[nH]1)C(=O)N2] |
| BIO | CDK | 2H-Indol-2-one, 6-bromo-3-[(3E)-1,3-dihydro-3- |
| | inhibitor | (hydroxyimino)-2H-indol-2-ylidene]-1,3- |
| | | dihydro-, (3Z)- |
| | | 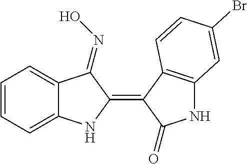 [SMILES: O=C1Nc2cc(Br)ccc2/C1=C1/Nc2ccccc2/C1=N\O] |
| AZD1080 | CDK | 1H-Indole-5-carbonitrile, 2-hydroxy-3-[5-(4- |
| | inhibitor | morpholinylmethyl)-2-pyridinyl]- |
| | | 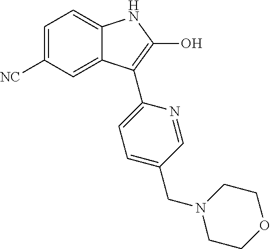 [SMILES: N#Cc1ccc2[nH]c(O)c(-c3ccc(CN4CCOCC4)cn3)c2c1] |
| Roscovitine | CDK | (R)-2-(6-(benzylamino)-9-isopropyl-9H-purin-2- |
| | inhibitor | ylamino)butan-1-ol |
| | | 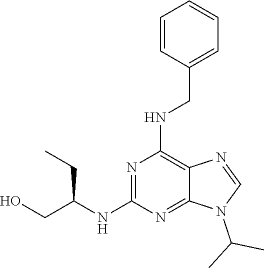 [SMILES: CC[C@H](CO)Nc1nc(NCc2ccccc2)c2ncn(C(C)C)c2n1] |
| Forskolin, | cAMP | (3R,4aR,5S,6S,6aS,10S,10aR,10bS)-6,10,10b- |
| FSK | activator | Trihydroxy-3,4a,7,7,10a-pentamethyl-1-oxo-3- |
| | | vinyldodecahydro-1H-benzo[f]chromen-5- |
| | | acetate |
| | | 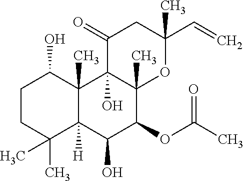 |

In particular embodiments, the ROCK inhibitor is Y2763 or a pharmaceutically acceptable salt thereof, the CDK inhibitor is SU9516 or a pharmaceutically acceptable salt thereof, and the cAMP inhibitor is forskolin or a pharmaceutically acceptable salt thereof.

For the purpose of delivery and absorption, effective amounts of a ROCK inhibitor, a CDK inhibitor, and a cAMP activator as described herein as an active ingredient can be formulated with a pharmaceutically acceptable carrier to form a composition in an appropriate form. Depending on the mode of administration, the composition of the present invention may contain about 0.1% to about 100% by weight of the active ingredient, wherein the percentage is calculated based on the total weight of the composition.

As used herein, “a pharmaceutically acceptable carrier” means that the carrier is compatible with an active ingredient in the composition, and preferably can stabilize said active ingredient and is safe to the receiving individual. Said carrier may be a diluent, vehicle, excipient, or matrix to the active ingredient. Said carrier may be a diluent, vehicle, excipient, or matrix to the active ingredient. Some examples of suitable excipients include lactose, glucose, sucrose, sorbitol, mannitol, starch, Arabic gum, calcium phosphate, alginate, tragacanth gum, gelatin, calcium silicate, microcrystalline cellulose, polyvinylpyrrolidone, cellulose, sterile water, saline, syrup and methylcellulose. The composition may additionally contain lubricants, such as talc, magnesium stearate and mineral oil; wetting agents; emulsifiers and suspending agents; preservatives, such as methyl and propyl hydroxybenzoates; sweeteners; and flavoring agents. Typically, a composition comprising as described herein can be in a form of a solution such as an aqueous solution i.e., a saline solution. The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be suspensions, lotions, solutions, sterilized injection fluid, and packaged powder. In certain embodiments, the composition of the present invention is formulated as a liquid injectable formulation which can be provided as a ready-to-use dosage form or as a reconstitutable stable powder.

In particular embodiments, an active ingredient or a composition thereof as described herein may be administered parenterally, more particularly, being administered through injection directly to a concerned region where myelin is lost or damaged. In some embodiments, the concerned region may include brain, optic nerves, and spinal cord.

In some embodiments, active ingredients or compositions thereof as described herein are administered simultaneously or sequentially.

In certain embodiments, active ingredients or compositions thereof as described herein are contained in the form of a kit that contains separate units for each of the active ingredients. In other embodiments, active ingredients or compositions thereof as described herein are combined together in the form of a single formulation.

The present invention features a cell-free therapy where a ROCK inhibitor, a CDK inhibitor and a cAMP activator are administered to a demyelinating lesion of a subject in need thereof which is effective in rescuing demyelination in vivo. As shown in the examples below, cuprizone causes demyelination of the cerebrum, cerebellar nuclei, brainstem, and especially the corpus callosum (CC); small molecules including Forskolin, Y27632, and Su9516 are directly injected into the corpus callosum of a cuprizone-induced demyelinated mouse brain model; and the results show that this combination of compounds promote remyelination in vivo after directly injecting into areas of the brain where myelin has been damaged. The method of the present invention is cell-free, which provides a convenient and promising way forward for treating demyelinating diseases.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

Oligodendrocytes are glial cells located in the central nervous system (CNS) that play essential roles in the transmission of nerve signals and in the neuroprotection of myelinated neurons. Dysfunction or loss of oligodendrocytes leads to demyelinating diseases such as multiple sclerosis (MS). To treat demyelinating diseases, the development of a therapy to promote remyelination is required. To pursue the development of a cell-free remyelination therapy in vivo, we used the cuprizone-induced demyelinated mouse model. The small molecules Y27632, SU9516, and forskolin (FSK) were directly injected into the demyelinated corpus callosum of the mouse brain. Excitingly, this combination of the small molecules rescued the demyelination phenotype within two weeks as observed by light and electron microscopy. These results provide the foundation for exploring the development of a treatment for demyelinating diseases via regenerative medicine.

### 1. Material and Methods

### 1.1. Cell Lines and Culture Condition

The human normal foreskin skin fibroblast primary cells (CRL-2097) were obtained from the ATCC. CRL-2097 cells were cultured at 37° C. with 5% CO2 in high glucose Dulbecco's Modified Eagle's Medium (HG-DMEM) (Life Technologies) supplemented with 10% fetal bovine serum (FBS) (Hyclone). Cell viability was determined by trypan blue staining. All of the cultures were free of mycoplasma. This study was approved by the Academia Sinica ethics committee (IRB number: AS-IRB-BM-17008, AS-IRB-BM-20038).

### 1.2 Cell Trans-Differentiation

Cells (1×10⁴ per well) were plated on 24-well plates. Cells were cultured in HG-DMEM (Life Technologies) supplemented with 10% fetal bovine serum (FBS) (Hyclone) for 2 days. Then the medium was replaced with DMEM/F12 (Gibco) with 1% N2 supplement, 1% B27 supplement (all from Life Technologies), 20 ng/ml Platelet-Derived Growth Factor AA (PDGF-AA), 20 ng/ml Epidermal Growth Factor (EGF), 20 ng/mL Basic Fibroblast Growth Factor (bFGF), 20 ng/ml Neurotrophin-3 (NT3) (all from Peprotech). Then the cells were treated with 3 mM valproic acid (Tocris) for 2 days, 10 μM Y27632 (LC Laboratories), 10 μM SU9516 (R&D Systems), and 10 μM Forskolin (FSK) (Tocris) for 1 day. The reagent is listed in supplementary Table S1 and Table S2.

### 1.3 Immunofluorescence Assay

Cells were fixed with 4% formaldehyde for 15 minutes at room temperature, and washed once with 1×Phosphate Buffered Saline (PBS). Cells were permeabilized with 0.3% Triton X-100 for 5 minutes, washed twice with 1×PBS, and blocked in 2% Bovine Serum Albumin (BSA) in PBS for 30 minutes. Then the cells were incubated with the Oligodendrocyte-specific marker 4 (04) antibody (Merck Millipore), Oligodendrocyte transcription factor (Olig2) antibody (Merck Millipore), Galactosylceramidase (GalC) antibody (Proteintech), Myelin Proteolipid Protein (PLP) antibody (Abcam), Myelin Basic Protein (MBP) antibody (Proteintech), and 4′,6-diamidino-2-phenylindole (DAPI) (Life Technologies) in blocking buffer (2% BSA in PBS) overnight at 4° C. The detailed information of the antibodies are listed in supplementary Table S3. Cells were washed twice in 1×PBS, followed by incubation in CF555 goat anti-rabbit secondary antibody (Life Technologies), CF488 goat anti-rabbit secondary antibody (Biotium), CF555 goat anti-mouse secondary antibody (Life Technologies), CF488 goat anti-mouse secondary antibody (Biotium), or CF555 goat anti-rat secondary antibody (Life Technologies) in blocking buffer for 1 hour in the dark at room temperature. Cells were washed twice in 1×PBS. Slides were mounted with ProLong Gold antifade reagents (Thermo Fisher). Fluorescence was analyzed using a fluorescence microscope (Axiovert 200 M, Zeiss).

### 1.4 Real-Time Quantitative PCR Assays

RNA was isolated from cells using the RNeasy Micro Kit (Qiagen) and was reverse-transcribed (400 ng). RT-PCR analysis was performed with the KAPA SYBR®FAST qPCR Kits (Kapa Biosystems) and the housekeeping gene Glyceraldehyde-3-Phosphate Dehydrogenase (GAPDH) was used for the endogenous reference. The primers are listed in the supplementary Table S4.

### 1.5 Flow Cytometry

Cells were harvested into 1.5 mL tubes filled with Accutase, washed 3 times with cold PBS, then blocked with 2% BSA in PBS for 15 minutes, and then stained with fluorescent-conjugated antibody A2B5 (Miltenyi Biotec) for 30 minutes. After 3 washes with PBS, cells were transferred to FACS buffer. A BD FACSCantoII (Franklin Lakes, New Jersey, United States) flow cytometer was used for the quantification of A2B5+ cells.

### 1.6 Western Blot Analysis

Cells were collected by scraping in lysis buffer (1% NP40, 50 mM Tris pH 8.0, 150 mM NaCl, 2 mM EDTA, 1 mM Na3VO4 and protease inhibitor cocktail (Sigma). Protein quantification was performed with the Biorad protein assay (Bio-Rad). Samples were boiled for 15 minutes at 100° C., 30 μg protein was loaded on 10% acrylamide gels, and electrophoresis was started at 90V for 15 minutes, then turned to 120V for 1 hour. Amersham Protran 0.45 μm nitrocellulose membranes were used for blotting. Blots were blocked for 30 minutes by shaking in 5% BSA in Phosphate Buffered Saline with Tween® 20 (PBST). Primary antibodies (NK2 Homeobox 2 (Nkx2.2), Proteintech) diluted in 5% BSA in PBST and incubated overnight at 4° C. on the shaker. Secondary antibodies were diluted in 5% BSA in PBST and culture for 1 hour at room temperature on the shaker. Detection of protein signals was performed with the UVP Biospectrum AC system (Jena, Thuringia, Germany).

1.7 Cuprizone-Induced Mouse Model Treated with Chemicals

8-10 week old C57/BL6 mice were used for the cuprizone-induced demyelination animal model. The mice were divided into three groups. The first group was fed the normal diet, which served as the positive control. For the two other groups, food was mixed with 0.2% (w/w) cuprizone as the diet. After 12 weeks of the cuprizone diet, mice were randomized into 2 groups. One group was injected with the chemical cocktail and the second group was injected with PBS. For the chemical and PBS injections, Zoletil and Ropum (1:1) were used for anesthesia, and the injection site was determined by a stereotaxic apparatus. Mice received 4 μL of the chemical mix (100 μM of Y27632, SU9516, and forskolin) and were subjected to a 10 minute deposition time at 0.8 L/min at the following coordinates: Bregma: +0.98 mm (anteroposterior axis), −1.75 mm (outer axis), −2.25 mm (vertical axis). One group received a 4 μL PBS injection as a control. At 14 days post-injection, transcardiac perfusion was prepared by pre-rinsing the tissue with 2% paraformaldehyde (PFA) in PBS, pH 7.4. Brains were explanted and then sectioned; immunohistochemical and electron microscopy analyses of the corpus callosum were performed (see below). The animal experiments were approved by the Academia Sinica committee (IACUC number: 18-02-1191).

### 1.8 Luxol Fast Blue

Corpus callosum slices were prepared by transcardial perfusion and embedded in epoxy resin embedding medium (Sigma). A microtome was used to cut the corpus callosum into 7 μm thick sections. Sections were stained with Luxol fast blue (LFB) solution. The area of Luxol fast blue was quantified by ImageJ analysis software.

### 1.9 Electron Microscopy

For electron microscopy analysis, transcardial perfusion was performed with fixative (4% paraformaldehyde and 2.5% glutaraldehyde in PBS, pH 7.4). The brains were explanted and kept overnight at 4° C. in fixative. The brains were cut into 1 mm slices and kept in fixative. The corpus callosum was cut into 1 mm×1 mm×2 mm tissue blocks and post-fixed in 1% osmium tetroxide (w/v). The blocks were dehydrated and embedded in Spurr's resin (Spurr Low Viscosity Embedding Kit; EMS®). After examining 0.5 μm semi-thin sections with toluidine blue staining for the presence of relevant regions, ultrathin sections (60 nm) were taken and counterstained with uranyl acetate and lead citrate, and then examined using a FEI Tecnai G2 F20 S-TWIN transmission electron microscope (Hillsboro, Oregon, United States).

### 1.10 Statistical Analysis

All statistical data are presented as the mean±standard deviation (S.D.) of at least three biological replicates. Statistical analysis was performed by unpaired two-tailed 1-tests and one-way ANOVA with GraphPad Prism 8.2.1 (GraphPad Software, CA, USA), where a p-value <0.05 was considered a significant difference. With Ordinary one-way ANOVA, the post hoc multiple comparisons tests were Tukey's multiple comparisons test.

### 2. Results

2.1 Chemical Reprogramming of Fibroblasts into an Oligodendrocyte-Like State in Vitro

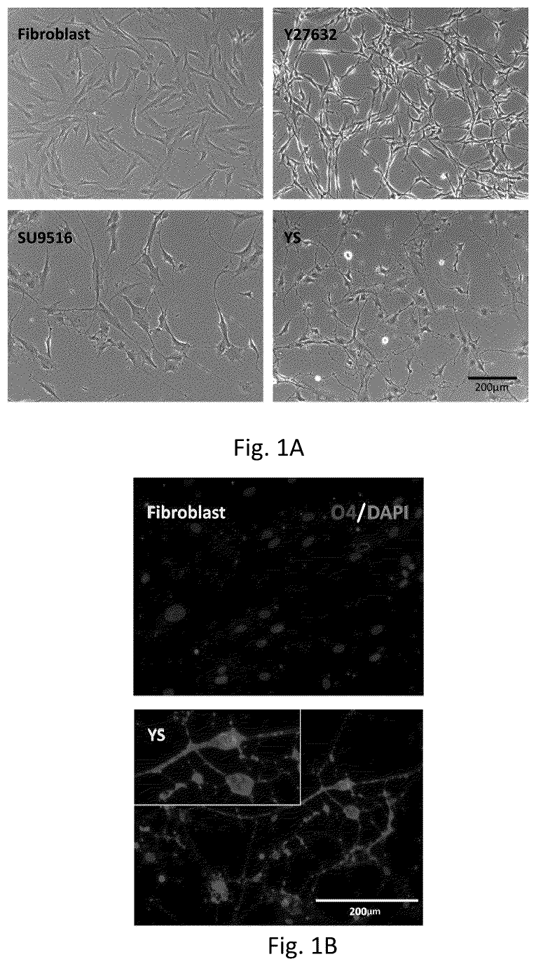

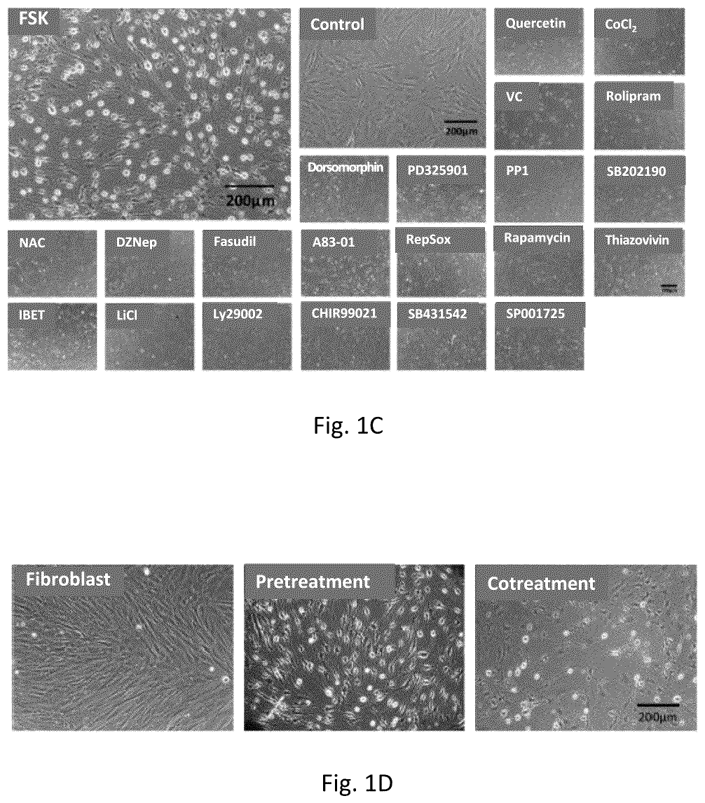

To find out which chemicals could convert fibroblasts into oligodendrocyte-like cells, we screened chemically-treated cells based on morphological changes that included the formation of a dendritic morphology of neural lineage cells and the dome-shaped morphology of the cell bodies of oligodendrocytes. In a preliminary screen, two chemicals, Y27632 and SU9516, were identified that may be involved in triggering the generation of oligodendrocyte-like cells from human primary fibroblasts (CRL2097). We observed that Y27632 and SU9516 treatment induced morphological changes in fibroblasts, becoming dendritic and dome-shaped (FIG. 1A). Therefore, we combined Y27632 and SU9516 into a cocktail to convert fibroblasts into an oligodendrocyte-like morphology (FIG. 1A). To characterize the induced oligodendrocyte-like cells, we performed immunofluorescence staining to analyze the expression of oligodendrocyte-specific markers. We initially analyzed the expression of oligodendrocyte-specific marker 4 (04) and found that the Y27632 and SU9516 treated groups had higher 04 expression compared to the solvent control group (FIG. 1B).

Before the next screening step, we noted that the chemical valproic acid (VPA), which is a histone deacetylase (HDAC) inhibitor commonly used in different reprogramming protocols, had been suggested to enhance reprogramming by modifying histones or other pathways in human and mouse fibroblasts [39-41]. Therefore, we added an initial treatment with VPA as a two-step treatment to facilitate the transformation of fibroblasts into oligodendrocyte-like cells. VPA has also been reported to enhance the chemical reprogramming of Schwann cells and neural precursor-like cells [42,43].

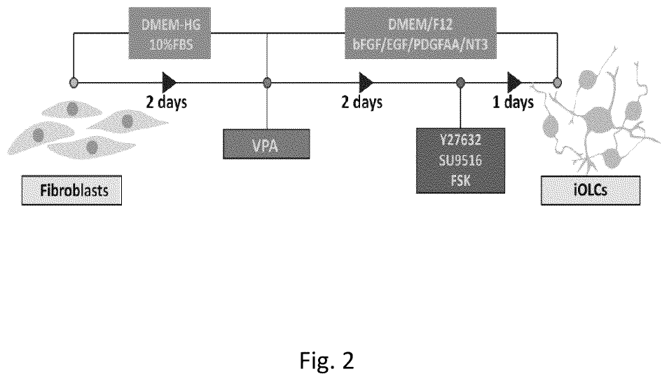

Next, to optimize the two-chemical cocktail, we performed an additional high-throughput screen using a chemical library of over 1,500 molecules purchased from Selleckchem®. The chemicals were rescreened for the ability to induce morphological changes in fibroblasts on top of a three-chemical formula including valproic acid (VPA), Y27632, and SU9516. Forskolin (FSK) was selected for its significant effect to induce morphological changes, with most cells exhibiting prominent oligodendrocyte-like morphology (FIG. 1C). FSK is an activator of cyclic adenosine monophosphate (cAMP), which has been shown to induce rat OPC maturation by up-regulating oligodendrocyte-specific proteins such as 2′,3′-Cyclic-nucleotide 3′-phosphodiesterase (CNP), Myelin Oligodendrocyte Glycoprotein (MOG), and Myelin Proteolipid Protein (PLP) [44]. Here, we found that FSK could increase the network complexity of oligodendrocyte-like cells under the combined action of VPA, Y27632, and SU9516. Cells were treated with VPA for two days, followed by treatment with Y27632, SU9516, and FSK for one day. We investigated co-processing VPA with the three other chemicals in the cocktail, however experiments showed that only pretreatment could improve the conversion efficiency (FIG. 1D). For cell trans-differentiation, our final optimized cocktail can transform cells into an oligodendrocyte-like state, in terms of morphology. In our case, we found that the four chemicals in combination could efficiently convert fibroblasts to oligodendrocyte-like cells within three days (FIG. 2).

### 2.2 Characterization of Chemically Induced Oligodendrocyte-Like Cells

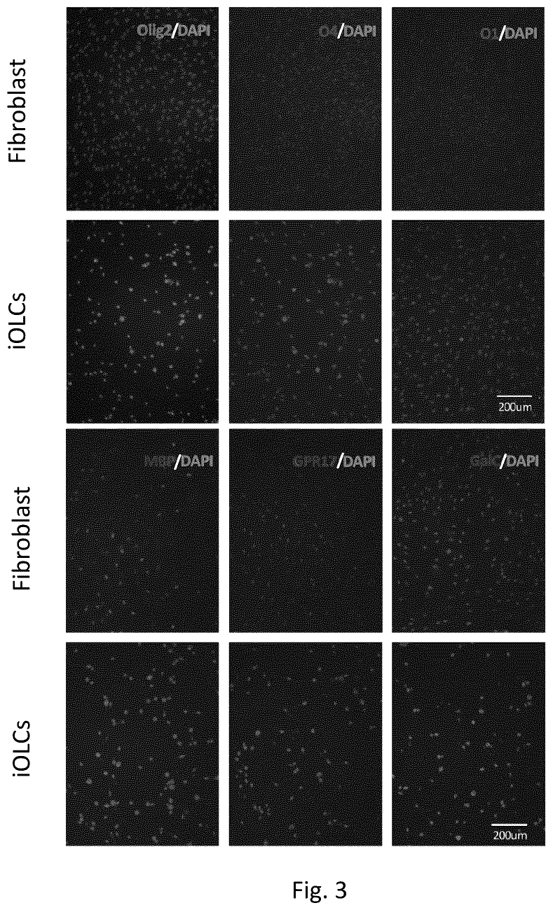

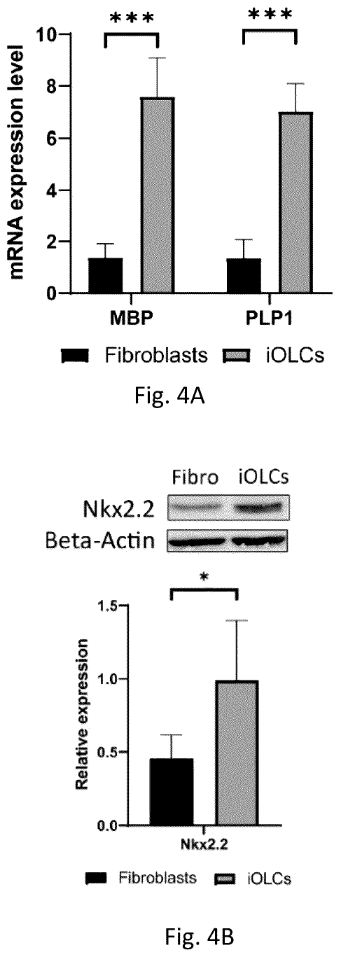

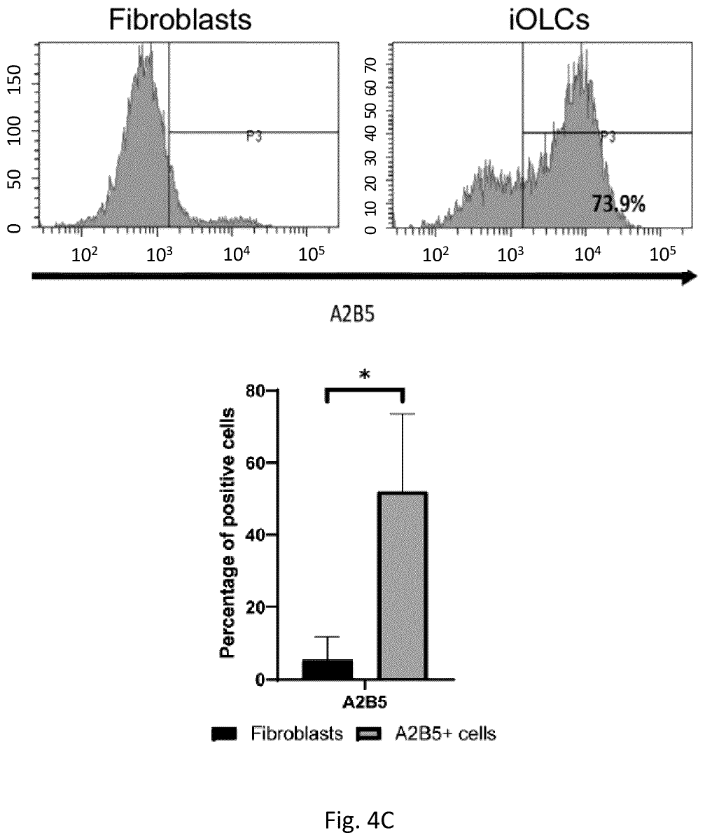

Next, to characterize the induced cells, we examined expression of markers in the iOLCs using immunofluorescence, quantitative reverse transcription-polymerase chain reaction (qRT-PCR), flow cytometry, and Western blotting. For the immunofluorescence experiments, we stained for the oligodendrocyte markers Olig2, Oligodendrocyte-specific marker 1 (01), and 04; Galactosylceramidase (GalC), G protein-coupled receptor 17 (GPR17), and myelin basic protein (MBP). As shown, O1, O4, Olig2, GPR17, GalC, and MBP were all expressed in the chemical cocktail-treated cells (FIG. 3). In addition, we found the expression levels of PLP1 and MBP were increased by qRT-PCR (FIG. 4A). The transcription factor NK2 Homeobox 2 (Nkx2.2) associated with OPC differentiation was up-regulated by Western blot analysis (FIG. 4B). The results showed that Nkx2.2, Proteolipid protein 1 (PLP1), and MBP were all highly expressed. To quantify the induced oligodendrocyte-like cells, we chose the surface antigen A2B5 of oligodendrocyte progenitor cells as the basis for quantification by flow cytometry, and the data indicated that approximately 50% of these cells were positive (FIG. 4C). Taken together, our data suggest that most fibroblasts are directed to trans-differentiate into the oligodendrocyte lineage cells.

### 2.3 Direct Chemical Injection Ameliorates Cuprizone-Induced Demyelination in Mice

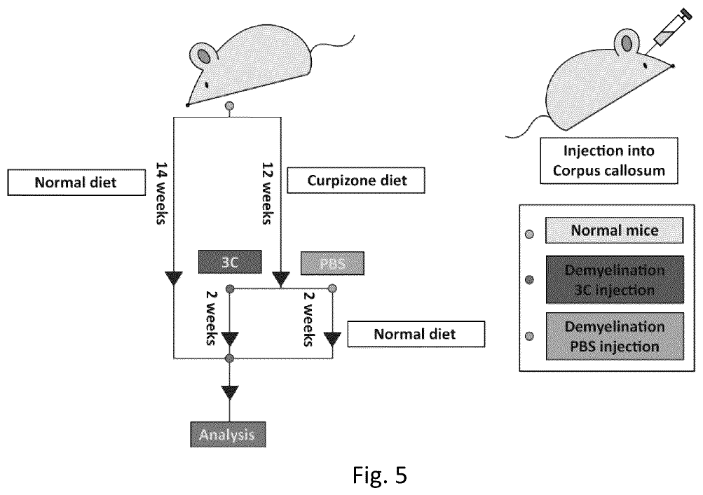

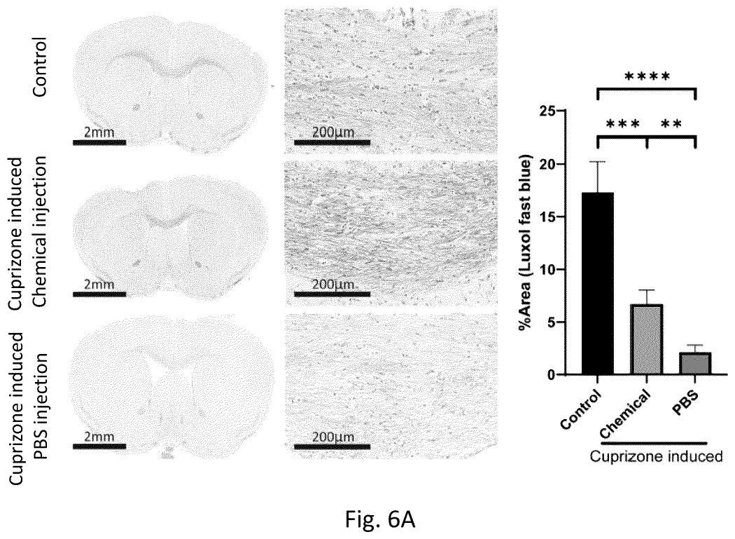

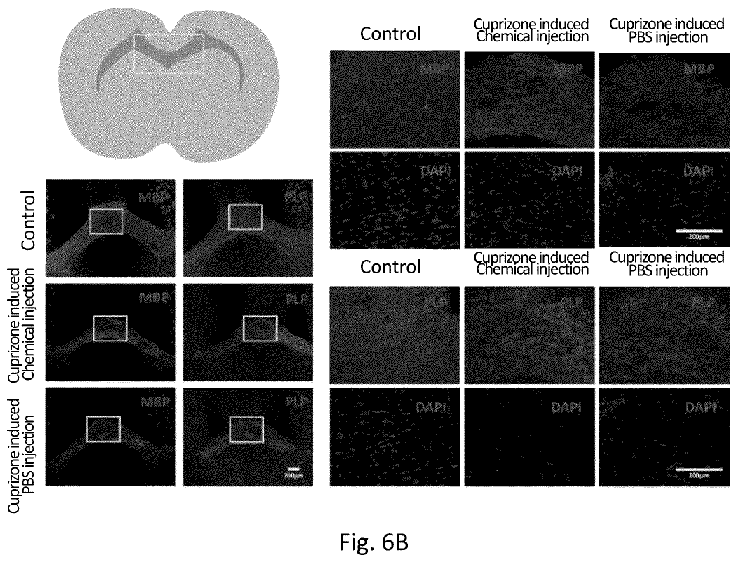

In our experiments, a chemical cocktail induced the formation of oligodendrocyte-like cells in vitro (FIGS. 1-4). To test the ability of the cocktail mixture to promote remyelination in vivo, we hypothesized that the chemical cocktail could directly promote remyelination in the corpus callosum. A murine model of cuprizone-induced demyelination was used to study the effects of the chemotherapy cocktail (FIG. 5). After 12 weeks of diet-induced demyelination by cuprizone, the 3 chemical (3C) cocktail (Y27632, FSK, and SU9516), and a PBS control, were injected directly into the mouse corpus callosum. After 2 weeks of recovery, Luxol fast blue staining revealed increased levels of remyelination in the chemical cocktail injection group as compared to the PBS control group (FIG. 6A). Likewise, in immunofluorescence staining, the protein levels of MBP and PLP were also higher in the chemical injection group compared to the PBS control group (FIG. 6B).

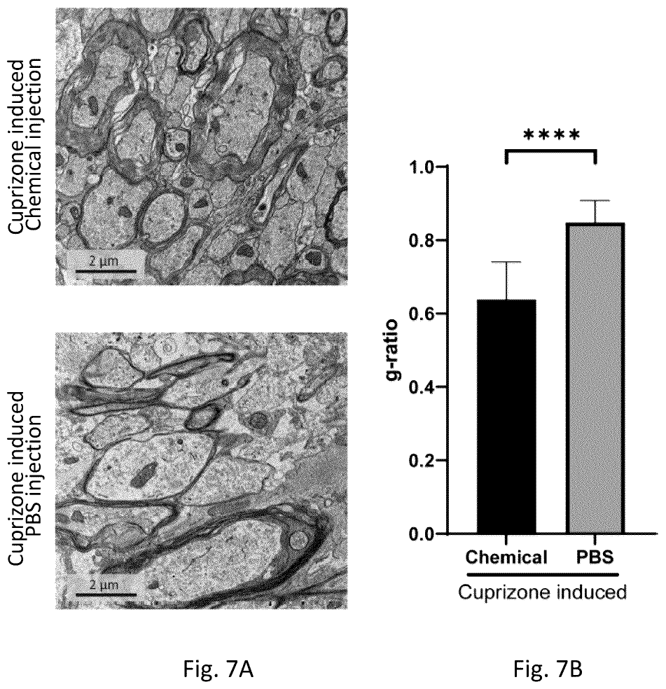

We next performed electron microscopy (EM) analyses to observe directly the different degrees of remyelination in detail. EM micrographs revealed the thicknesses of myelin sheaths (FIG. 7A). To quantify the level of remyelination, a g-ratio was calculated, and a lower g-ratio was observed in the chemical injected group as compared to the PBS control group (FIG. 7B).

### 3. Discussion

We have discovered a chemical formulation to convert human fibroblasts into iOLCs within 3 days. These iOLCs displayed OPC-like morphology and expressed the main molecular features of OPCs. Previous studies indicated that transcription factor-mediated conversion of mouse fibroblasts into OPCs can be performed by the activity of SRY-Box Transcription Factor 10 (Sox 10), Olig2, and zinc finger protein 536 (Zfp536)/NK6 Homeobox 2 (Nkx6.2) in 14 to 21 days [25,26]. In another chemical conversion procedure, Liu et al. published a two-step protocol. At first, they converted mouse fibroblasts into an unstable intermediate cell population of neural lineage cells using M9 medium, and then they redirected the cells by tuning specific culture signals towards the OPC state [45]. The M9 cocktail, including 9 small molecules CHIR99021, LDN193189, A83-01, Hh-Ag1.5, retinoic acid (RA), SMER28, RG108, SB431542 and Parnate, can convert mouse fibroblasts into a transition state and then the media containing LDN193189, SB431542, retinoic acid and Sonic hedgehog can be employed to convert these cells into the OPC-like state in 7 days [45]. The conversion rate was about 25% as quantified by Olig2 [45]. In contrast to the induction approach by Liu et al. in mouse cells, our chemical method includes three major small molecules, forskolin, Y27632, and SU9516 that can convert human cells, which are very different in composition. Our results suggest that the underlying molecular/cellular mechanisms of conversion are likely very different. Our approach generates OPC-like cells more directly and faster than the M9 medium induction method. In our experiments, we treated the cells with VPA, a histone deacetylase inhibitor, for 2 days to eliminate epigenetic signatures and then with Y27632, forskolin, and Su9516 for 1 day for OPC-like cell induction in vitro. Y27632 is a Rho-related kinase (ROCK) inhibitor that can promote stem cell proliferation [46], increase neuroprotection during development [47], and has anti-apoptotic functions [46,48]. Notably, forskolin, a cAMP activator, has always played a critical role in the field of the reprogramming of neural lineage cells [49-51]. These factors may also support the conversion of fibroblasts into neural cells. In addition, in our cocktail, it can be inferred that SU9516, a Cyclin-dependent kinase 2 (Cdk2) inhibitor, plays a key role in converting human fibroblasts into OPC-like cells. Previous studies reported that Cdk2 is not essential for mice and the oligodendrocytes in Cdk2 knockout mice have higher differentiation and better remyelination abilities [53]. SU9516 has also been reported to improve muscle function by increasing α7β1 integrin of skeletal muscle in the Duchenne Muscular Dystrophy (DMD) mouse model [54]. This may be related to our findings that inhibition of Cdk2 can convert human fibroblasts into OLCs and promote remyelination in the demyelinated central nervous system.

Although autologous-derived cell transplantation is considered a strategy for the treatment of myelin degeneration, for the practical application of the protocols, there are still many problems to be solved. For example, induced cells are difficult to control due to their heterogeneity, and transplanted cells have low viability after integration. In addition, current viral reprogramming methods for generating iOLCs are generally inefficient, with only around 2%-15% of the cells being reprogrammed, but, in the future, an increase in viral titer may increase cell reprogramming efficiency [25,26]. In contrast, chemically generated mouse iOPCs can achieve higher reprogramming efficiencies up to a level of approximately 25% Olig2+ and 13% Nkx2.2+ cells [45]. Here, we converted human fibroblasts into iOLCs using a cocktail of small molecules in vitro, measuring 50% or more A2B5+ cells by flow cytometry, achieving a significant increase in reprogramming efficiency. Small molecules can enter cells in a short period of time and do not have the problems of insertional mutagenesis caused by viral infections, immune rejection, and are low in cost. Therefore, the rescue of myelin degeneration using a cocktail of small molecules is currently the most economical and convenient cell-free therapeutic method for experimental cell reprogramming in vitro, and for pursuing the development of an in vivo drug therapy.

In previous studies, the majority of mice with myelin degeneration in the cuprizone-induced model were treated with a single agent. These single treatment modalities have provided insight into the role of a single message on remyelination in vivo. A non-steroidal anti-inflammatory drug (NSAID), Indomethacin, is a glycogen synthase kinase 3 beta (GSK3β) inhibitor and has been shown to promote differentiation of primary oligodendrocytes, and promote remyelination in a 10-week cuprizone-induced demyelination model [33]. BLZ945, a colony stimulating factor 1 (CSF-1) receptor kinase inhibitor, enhances remyelination by modulating neuroinflammation in a 5-week cuprizone-induced model [34]. Linagliptin, a dipeptidyl peptidase (DPP)-4 inhibitor for the treatment of adult type 2 diabetes, has been reported to attenuate 4-week cuprizone-induced demyelination by modulating AMP-activated protein kinase/Sirtuin 1 (AMPK/SIRT1) and Janus kinase 2/Signal Transducer And Activator Of Transcription 3/nuclear factor kappa-light-chain-enhancer of activated B cells (JAK2/STAT3/NF-κB) signaling pathways [35]. Ginkgo biloba extract is a traditional Chinese herbal medicine that has also been shown to have neuroprotective, anti-inflammatory, antioxidant, and anti-apoptotic effects [36]. Bilobalide, a component present in the Ginkgo biloba extract, has been reported to inhibit microglia-associated neuro-inflammation and promote remyelination in a cuprizone-induced 6-week model [36]. However, there are two types of cuprizone treatment methods to induce demyelination. The first one is to treat the mice with cuprizone for 12- to 13-weeks to obtain chronically demyelinated lesions [37]. Due to the long-term treatment of cuprizone, the ability for myelin repair endogenously is limited [55,56]. The other model is acute demyelination induced by only feeding the mice with cuprizone for 5-6 weeks. However, if animals were given normal chow after week 5, spontaneous endogenous remyelination occurred efficiently. In brief, the short-term cuprizone-model can induce acute demyelination, which is more suitable for the study of immunomodulation in MS. Therefore, acute cuprizone-induced demyelination should not be used to study the effects of factors on remyelination. In order to study remyelination in a non-supportive environment, a chronic cuprizone model must be selected [37,56,57]. Therefore, to avoid the autologous myelin repair effects on the experimental results, we treated with cuprizone for 12 weeks to obtain chronic long-term demyelinating lesions and found that our small molecule formulation can effectively rescue cuprizone-induced degeneration of myelin in the mouse corpus callosum.

Although there might be some glial activation in both the cuprizone treated PBS group and 3C cocktail treated groups, the 3C cocktail treated group displayed oligodendrocyte recovery at a level significantly higher than control as shown by the Luxol fast blue staining (FIG. 6A), Olig2 PLP (FIG. 6B) and MBP (FIG. 6B).

### 4. Conclusions

In this art, it is generally understood that compounds that can induce cellular reprograming in vitro would not necessarily lead to the same efficacy in vivo since it may diffuse in the body. Here, we report that a small molecule cocktail has the potential to reprogram human fibroblasts into an oligodendrocyte-like state based on displaying a similar morphology and specific oligodendrocyte markers; and injection of the chemical cocktail promoted the process of remyelination in the corpus callosum of cuprizone-induced demyelinated mice. Combined, our results surprisingly show that a chemical cocktail can generate oligodendrocyte-like cells and rescue demyelination in vivo. Our research can provide a starting point for the development of a procedure to use regenerative medicine as a potential cell-free therapy for MS treatment in the future.

### REFERENCE

1. Allen, N.J.; Barres, B. A. Neuroscience: Glia-more than just brain glue. Nature 2009, 457, 675-677, doi:10.1038/457675a.
2. Marinelli, C.; Bertalot, T.; Zusso, M.; Skaper, S.D.; Giusti, P. Systematic Review of Pharmacological Properties of the Oligodendrocyte Lineage. Front Cell Neurosci 2016, 10, 27, doi:10.3389/fncel.2016.00027.
3. Kier, L. B.; Tombes, R. M. Proton hopping: a proposed mechanism for myelinated axon nerve impulses. Chem Biodivers 2013, 10, 596-599, doi: 10.1002/cbdv.201200417.
4. Rouach, N.; Koulakoff, A.; Abudara, V.; Willecke, K.; Giaume, C. Astroglial metabolic networks sustain hippocampal synaptic transmission. Science 2008, 322, 1551-1555, doi: 10.1126/science.1164022.
5. Niu, J.; Li, T.; Yi, C.; Huang, N.; Koulakoff, A.; Weng, C.; Li, C.; Zhao, C. J.; Giaume, C.; Xiao, L. Connexin-based channels contribute to metabolic pathways in the oligodendroglial lineage. J Cell Sci 2016, 129, 1902-1914, doi: 10.1242/jcs.178731.
6. Morrison, B. M.; Lee, Y.; Rothstein, J. D. Oligodendroglia: metabolic supporters of axons. Trends Cell Biol 2013, 23, 644-651, doi:10.1016/j.tcb.2013.07.007.
7. Hirrlinger, J.; Nave, K. A. Adapting brain metabolism to myelination and long-range signal transduction. Glia 2014, 62, 1749-1761, doi: 10.1002/glia.22737.
8. Bergles, D. E.; Richardson, W. D. Oligodendrocyte Development and Plasticity. Cold Spring Harb Perspect Biol 2015, 8, a020453, doi: 10.1101/cshperspect.a020453.
9. Bradl, M.; Lassmann, H. Oligodendrocytes: biology and pathology. Acta Neuropathol 2010, 119, 37-53, doi: 10.1007/s00401-009-0601-5.
10. Skaper, S.D. Oligodendrocyte precursor cells as a therapeutic target for demyelinating diseases. Prog Brain Res 2019, 245, 119-144, doi: 10.1016/bs.pbr.2019.03.013.
11. Inglese, M. Multiple sclerosis: new insights and trends. AJNR Am J Neuroradiol 2006, 27, 954-957.
12. Najm, F. J.; Madhavan, M.; Zaremba, A.; Shick, E.; Karl, R. T.; Factor, D. C.; Miller, T. E.; Nevin, Z. S.; Kantor, C.; Sargent, A.; et al. Drug-based modulation of endogenous stem cells promotes functional remyelination in vivo. Nature 2015, 522, 216-220, doi: 10.1038/nature14335.
13. Imani, A.; Golestani, M. Cost-utility analysis of disease-modifying drugs in relapsing-remitting multiple sclerosis in Iran. Iran J Neurol 2012, 11, 87-90.
14. Gallo, P.; Van Wijmeersch, B.; Paradig, M. S. G. Overview of the management of relapsing-remitting multiple sclerosis and practical recommendations. Eur J Neurol 2015, 22 Suppl 2, 14-21, doi: 10.1111/ene.12799.
15. Compston, A.; Coles, A. Multiple sclerosis. Lancet 2008, 372, 1502-1517, doi: 10.1016/S0140-6736(08)61620-7.
16. Kawabata, S.; Takano, M.; Numasawa-Kuroiwa, Y.; Itakura, G.; Kobayashi, Y.; Nishiyama, Y.; Sugai, K.; Nishimura, S.; Iwai, H.; Isoda, M.; et al. Grafted Human iPS Cell-Derived Oligodendrocyte Precursor Cells Contribute to Robust Remyelination of Demyelinated Axons after Spinal Cord Injury. Stem Cell Reports 2016, 6, 1-8, doi: 10.1016/j.stemcr.2015.11.013.
17. Tontsch, U.; Archer, D. R.; Dubois-Dalcq, M.; Duncan, I. D. Transplantation of an oligodendrocyte cell line leading to extensive myelination. Proc Natl Acad Sci USA 1994, 91, 11616-11620.
18. Groves, A. K.; Barnett, S. C.; Franklin, R. J.; Crang, A. J.; Mayer, M.; Blakemore, W. F.; Noble, M. Repair of demyelinated lesions by transplantation of purified O-2A progenitor cells. Nature 1993, 362, 453-455, doi: 10.1038/362453a0.
19. Guarino, A. T.; Mckinnon, R. D. Reprogramming cells for brain repair. Brain Sci 2013, 3, 1215-1228, doi: 10.3390/brainsci3031215.
20. Ogawa, S.; Tokumoto, Y.; Miyake, J.; Nagamune, T. Immunopanning selection of A2B5-positive cells increased the differentiation efficiency of induced pluripotent stem cells into oligodendrocytes. Neurosci Lett 2011, 489, 79-83, doi: 10.1016/j.neulet.2010.11.070.
21. Biswas, D.; Jiang, P. Chemically Induced Reprogramming of Somatic Cells to Pluripotent Stem Cells and Neural Cells. Int J Mol Sci 2016, 17, 226, doi: 10.3390/ijms17020226.
22. Aharonowiz, M.; Einstein, O.; Fainstein, N.; Lassmann, H.; Reubinoff, B.; Ben-Hur, T. Neuroprotective effect of transplanted human embryonic stem cell-derived neural precursors in sclerosis. PLOS One 2008, 3, e3145, an animal model of multiple doi: 10.1371/journal.pone.0003145.
23. Mikaeili Agah, E.; Parivar, K.; Joghataei, M. T. Therapeutic effect of transplanted human Wharton's jelly stem cell-derived oligodendrocyte progenitor cells (hWJ-MSC-derived OPCs) in an animal model of multiple sclerosis. Mol Neurobiol 2014, 49, 625-632, doi: 10.1007/s12035-013-8543-2.
24. Tsui, Y. P.; Lam, G.; Wu, K. L.; Li, M. T.; Tam, K. W.; Shum, D. K.; Chan, Y. S. Derivation of Oligodendrocyte Precursors from Adult Bone Marrow Stromal Cells for Remyelination Therapy. Cells 2021, 10, doi: 10.3390/cells10082166.
25. Najm, F. J.; Lager, A. M.; Zaremba, A.; Wyatt, K.; Caprariello, A. V.; Factor, D. C.; Karl, R. T.; Maeda, T.; Miller, R. H.; Tesar, P. J. Transcription factor-mediated reprogramming of fibroblasts to expandable, myelinogenic oligodendrocyte progenitor cells. Nat Biotechnol 2013, 31, 426-433, doi:10.1038/nbt.2561.
26. Yang, N.; Zuchero, J. B.; Ahlenius, H.; Marro, S.; Ng, Y. H.; Vierbuchen, T.; Hawkins, J. S.; Geissler, R.; Barres, B. A.; Wernig, M. Generation of oligodendroglial cells by direct lineage conversion. Nat Biotechnol 2013, 31, 434-439, doi: 10.1038/nbt.2564.
27. Bulic-Jakus, F.; Katusic Bojanac, A.; Juric-Lekic, G.; Vlahovic, M.; Sincic, N. Teratoma: from spontaneous tumors to the pluripotency/malignancy assay. Wiley Interdiscip Rev Dev Biol 2016, 5, 186-209, doi: 10.1002/wdev.219.
28. Alsanie, W. F.; Niclis, J. C.; Petratos, S. Human embryonic stem cell-derived oligodendrocytes: protocols and perspectives. Stem Cells Dev 2013, 22, 2459-2476, doi: 10.1089/scd.2012.0520.
29. Wang, S.; Bates, J.; Li, X.; Schanz, S.; Chandler-Militello, D.; Levine, C.; Maherali, N.; Studer, L.; Hochedlinger, K.; Windrem, M.; et al. Human iPSC-derived oligodendrocyte progenitor cells can myelinate and rescue a mouse model of congenital hypomyelination. Cell Stem Cell 2013, 12, 252-264, doi:10.1016/j.stem.2012.12.002.
30. Liu, C.; Hu, X.; Li, Y.; Lu, W.; Li, W.; Cao, N.; Zhu, S.; Cheng, J.; Ding, S.; Zhang, M. Conversion of mouse fibroblasts into oligodendrocyte progenitor-like cells through a chemical approach. J Mol Cell Biol 2019, 11, 489-495, doi: 10.1093/jmcb/mjy088.
31. Evangelista, A. F.; Soares, M. B. P.; Villarreal, C. F. Cell-free therapy: a neuroregenerative approach to sensory neuropathy? Neural Regen Res 2019, 14, 1383-1384, doi: 10.4103/1673-5374.253522.
32. Chen, Y. R.; Lai, P. L.; Chien, Y.; Lee, P. H.; Lai, Y. H.; Ma, H. I.; Shiau, C. Y.; Wang, K. C. Improvement of Impaired Motor Functions by Human Dental Exfoliated Deciduous Teeth Stem Cell-Derived Factors in a Rat Model of Parkinson's Disease. Int J Mol Sci 2020, 21, doi:10.3390/ijms21113807.
33. Preisner, A.; Albrecht, S.; Cui, Q. L.; Hucke, S.; Ghelman, J.; Hartmann, C.; Taketo, M. M.; Antel, J.; Klotz, L.; Kuhlmann, T. Non-steroidal anti-inflammatory drug indometacin enhances endogenous remyelination. Acta Neuropathol 2015, 130, 247-261, doi: 10.1007/s00401-015-1426-Z.
34. Beckmann, N.; Giorgetti, E.; Neuhaus, A.; Zurbruegg, S.; Accart, N.; Smith, P.; Perdoux, J.; Perrot, L.; Nash, M.; Desrayaud, S.; et al. Brain region-specific enhancement of remyelination and prevention of demyelination by the CSFIR kinase inhibitor BLZ945. Acta Neuropathol Commun 2018, 6, 9, doi: 10.1186/s40478-018-0510-8.
35. Elbaz, E. M.; Senousy, M. A.; El-Tanbouly, D. M.; Sayed, R. H. Neuroprotective effect of linagliptin against cuprizone-induced demyelination and behavioural dysfunction in mice: A pivotal role of AMPK/SIRT1 and JAK2/STAT3/NF-kappaB signalling pathway modulation. Toxicol Appl Pharmacol 2018, 352, 153-161, doi: 10.1016/j.taap.2018.05.035.
36. Sui, R. X.; Miao, Q.; Wang, J.; Wang, Q.; Song, L. J.; Yu, J. W.; Cao, L.; Xiao, W.; Xiao, B. G.; Ma, C. G. Protective and therapeutic role of Bilobalide in cuprizone-induced demyelination. Int Immunopharmacol 2019, 66, 69-81, doi: 10.1016/j.intimp.2018.09.041.
37. Zhan, J.; Mann, T.; Joost, S.; Behrangi, N.; Frank, M.; Kipp, M. The Cuprizone Model: Dos and Do Nots. Cells 2020, 9, doi: 10.3390/cells9040843.
38. Xing, Y. L.; Roth, P. T.; Stratton, J. A.; Chuang, B. H.; Danne, J.; Ellis, S. L.; Ng, S. W.; Kilpatrick, T. J.; Merson, T. D. Adult neural precursor cells from the subventricular zone contribute significantly to oligodendrocyte regeneration and remyelination. J Neurosci 2014, 34, 14128-14146, doi: 10.1523/JNEUROSCI.3491-13.2014.
39. Huangfu, D.; Maehr, R.; Guo, W.; Eijkelenboom, A.; Snitow, M.; Chen, A. E.; Melton, D. A. Induction of pluripotent stem cells by defined factors is greatly improved by small-molecule compounds. Nat Biotechnol 2008, 26, 795-797, doi: 10.1038/nbt1418.
40. Ehashi, T.; Suzuki, N.; Ando, S.; Sumida, K.; Saito, K. Effects of valproic acid on gene expression during human embryonic stem cell differentiation into neurons. J Toxicol Sci 2014, 39, 383-390, doi:10.2131/jts.39.383.
41. Duan, Q.; Li, S.; Wen, X.; Sunnassee, G.; Chen, J.; Tan, S.; Guo, Y. Valproic Acid Enhances Reprogramming Efficiency and Neuronal Differentiation on Small Molecules Staged-Induction Neural Stem Cells: Suggested Role of mTOR Signaling. Front Neurosci 2019, 13, 867, doi: 10.3389/fnins.2019.00867.
42. Thoma, E. C.; Merkl, C.; Heckel, T.; Haab, R.; Knoflach, F.; Nowaczyk, C.; Flint, N.; Jagasia, R.; Jensen Zoffmann, S.; Truong, H. H.; et al. Chemical conversion of human fibroblasts into functional Schwann cells. Stem Cell Reports 2014, 3, 539-547, doi: 10.1016/j.stemcr.2014.07.014.
43. Raciti, M.; Granzotto, M.; Duc, M. D.; Fimiani, C.; Cellot, G.; Cherubini, E.; Mallamaci, A. Reprogramming fibroblasts to neural-precursor-like cells by structured overexpression of pallial patterning genes. Mol Cell Neurosci 2013, 57, 42-53, doi: 10.1016/j.mcn.2013.10.004.
44. Joubert, L.; Foucault, I.; Sagot, Y.; Bernasconi, L.; Duval, F.; Alliod, C.; Frossard, M. J.; Pescini Gobert, R.; Curchod, M. L.; Salvat, C.; et al. Chemical inducers and transcriptional markers of oligodendrocyte differentiation. J Neurosci Res 2010, 88, 2546-2557, doi: 10.1002/jnr.22434.
45. Liu, C.; Hu, X.; Li, Y.; Lu, W.; Li, W.; Cao, N.; Zhu, S.; Cheng, J.; Ding, S.; Zhang, M. Conversion of mouse fibroblasts into oligodendrocyte progenitor-like cells through a chemical approach. J Mol Cell Biol 2019, doi: 10.1093/jmcb/mjy088.
46. Compagnucci, C.; Barresi, S.; Petrini, S.; Billuart, P.; Piccini, G.; Chiurazzi, P.; Alfieri, P.; Bertini, E.; Zanni, G. Rho Kinase Inhibition Is Essential During In Vitro Neurogenesis and Promotes Phenotypic Rescue of Human Induced Pluripotent Stem Cell-Derived Neurons With Oligophrenin-1 Loss of Function. Stem Cells Transl Med 2016, 5, 860-869, doi:10.5966/sctm.2015-0303.
47. Ding, J.; Yu, J. Z.; Li, Q. Y.; Wang, X.; Lu, C. Z.; Xiao, B. G. Rho kinase inhibitor Fasudil induces neuroprotection and neurogenesis partially through astrocyte-derived G-CSF. Brain Behav Immun 2009, 23, 1083-1088, doi: 10.1016/j.bbi.2009.05.002.
48. Paintlia, A. S.; Paintlia, M. K.; Singh, A. K.; Singh, I. Inhibition of rho family functions by lovastatin promotes myelin repair in ameliorating experimental autoimmune encephalomyelitis. Mol Pharmacol 2008, 73, 1381-1393, doi: 10.1124/mol. 107.044230.
49. Li, X.; Zuo, X.; Jing, J.; Ma, Y.; Wang, J.; Liu, D.; Zhu, J.; Du, X.; Xiong, L.; Du, Y.; et al. Small-Molecule-Driven Direct Reprogramming of Mouse Fibroblasts into Functional Neurons. Cell Stem Cell 2015, 17, 195-203, doi: 10.1016/j.stem.2015.06.003.
50. Hu, W.; Qiu, B.; Guan, W.; Wang, Q.; Wang, M.; Li, W.; Gao, L.; Shen, L.; Huang, Y.; Xie, G.; et al. Direct Conversion of Normal and Alzheimer's Disease Human Fibroblasts into Neuronal Cells by Small Molecules. Cell Stem Cell 2015, 17, 204-212, doi: 10.1016/j.stem.2015.07.006.
51. Gao, L.; Guan, W.; Wang, M.; Wang, H.; Yu, J.; Liu, Q.; Qiu, B.; Yu, Y.; Ping, Y.; Bian, X.; et al. Direct Generation of Human Neuronal Cells from Adult Astrocytes by Small Molecules. Stem Cell Reports 2017, 8, 538-547, doi: 10.1016/j.stemcr.2017.01.014.
52. Berthet, C.; Aleem, E.; Coppola, V.; Tessarollo, L.; Kaldis, P. Cdk2 knockout mice are viable. Curr Biol 2003, 13, 1775-1785, doi: 10.1016/j.cub.2003.09.024.
53. Caillava, C.; Vandenbosch, R.; Jablonska, B.; Deboux, C.; Spigoni, G.; Gallo, V.; Malgrange, B.; Baron-Van Evercooren, A. Cdk2 loss accelerates precursor differentiation and remyelination in the adult central nervous system. J Cell Biol 2011, 193, 397-407, doi: 10.1083/jcb.201004146.
54. Sarathy, A.; Wuebbles, R. D.; Fontelonga, T. M.; Tarchione, A. R.; Mathews Griner, L. A.; Heredia, D. J.; Nunes, A. M.; Duan, S.; Brewer, P. D.; Van Ry, T.; et al. SU9516 Increases alpha7beta1 Integrin and Ameliorates Disease Progression in the mdx Mouse Model of Duchenne Muscular Dystrophy. Mol Ther 2017, 25, 1395-1407, doi: 10.1016/j.ymthe.2017.03.022.
55. Slowik, A.; Schmidt, T.; Beyer, C.; Amor, S.; Clarner, T.; Kipp, M. The sphingosine 1-phosphate receptor agonist FTY720 is neuroprotective after cuprizone-induced CNS demyelination. Br J Pharmacol 2015, 172, 80-92, doi:10.1111/bph.12938.
56. Lindner, M.; Fokuhl, J.; Linsmeier, F.; Trebst, C.; Stangel, M. Chronic toxic demyelination in the central nervous system leads to axonal damage despite remyelination. Neurosci Lett 2009, 453, 120-125, doi:10.1016/j.neulet.2009.02.004.
57. Kipp, M. Remyelination strategies in multiple sclerosis: a critical reflection. Expert Rev Neurother 2016, 16, 1-3, doi: 10.1586/14737175.2016.1116387.

What is claimed is:

1. A method for treating a demyelinating condition in a subject in need thereof, comprising administering to the subject therapeutically effective amounts of a Rho-associated protein kinase (ROCK) inhibitor, a cyclin-dependent kinase (CDK) inhibitor, and a cyclic adenosine monophosphate (cAMP) activator.

2. The method of claim 1, wherein the amounts of the ROCK inhibitor, the CDK inhibitor and the cAMP inhibitor are effective in increasing a level of remyelination and/or decreasing a level of demyelination in the subject.

3. The method of claim 1, wherein the ROCK inhibitor, the CDK inhibitor and the cAMP inhibitor are administrated into a demyelinating lesion in the subject where myelin is lost or damaged.

4. The method of claim 1, wherein the ROCK inhibitor, the CDK inhibitor and the cAMP inhibitor are injected to the brain of the subject.

5. The method of claim 1, wherein the ROCK inhibitor, the CDK inhibitor and the cAMP inhibitor are small molecules.

6. The method of claim 1, wherein the ROCK inhibitor is Y27632 or a pharmaceutically acceptable salt, the CDK inhibitor is SU9516 or a pharmaceutically acceptable salt, and the cAMP inhibitor is forskolin or a pharmaceutically acceptable salt.

7. The method of claim 1, wherein the ROCK inhibitor, the CDK inhibitor and the cAMP inhibitor are administered simultaneously or sequentially.

8. The method of claim 1, wherein the ROCK inhibitor, the CDK inhibitor and the cAMP inhibitor are formulated in a composition.

9. The method of claim 1, wherein the demyelinating condition is an inflammatory demyelinating condition.

10. The method of claim 1, wherein the demyelinating condition occurs in multiple sclerosis (MS), acute hemorrhagic inflammatory disease (AHL), cerebral palsy, acute-disseminated encephalomyelitis (ADEM), central pontine myelinolysis, progressive multifocal leukoencephalopathy, congenital leukodystrophies, Parkinson's disease, Huntington's disease, schizophrenia.

11. The method of claim 6, wherein the ROCK inhibitor, the CDK inhibitor and the cAMP inhibitor are administered simultaneously or sequentially.

12. The method of claim 6, wherein the ROCK inhibitor, the CDK inhibitor and the cAMP inhibitor are formulated in a composition.

13. The method of claim 6, wherein the demyelinating condition is an inflammatory demyelinating condition.

14. The method of claim 6, wherein the demyelinating condition occurs in multiple sclerosis (MS), acute hemorrhagic inflammatory disease (AHL), cerebral palsy, acute-disseminated encephalomyelitis (ADEM), central pontine myelinolysis, progressive multifocal leukoencephalopathy, congenital leukodystrophies, Parkinson's disease, Huntington's disease, schizophrenia.

15. The method of claim 6, wherein the ROCK inhibitor is Y27632, the CDK inhibitor is SU9516, and the cAMP inhibitor is forskolin.

16. The method of claim 15, wherein the Y27632, SU9516, and forskolin are administered simultaneously or sequentially.

17. The method of claim 15, wherein the Y27632, SU9516, and forskolin are formulated in a composition.

18. The method of claim 15, where in the demyelinating condition is an inflammatory demyelinating condition.

19. The method of claim 15, wherein the demyelinating condition occurs in multiple sclerosis (MS), acute hemorrhagic inflammatory disease (AHL), cerebral palsy, acute-disseminated encephalomyelitis (ADEM), central pontine myelinolysis, progressive multifocal leukoencephalopathy, congenital leukodystrophies, Parkinson's disease, Huntington's disease, schizophrenia.
